(19) United States
(12) Patent Application Publication
(10) Pub. No.: US 2019/0160148 A1
(43) Pub. Date: May 30, 2019

(54) COMBINATION OF PEMBROLIZUMAB AND ABEMACICLIB FOR THE TREATMENT OF CANCER

(71) Applicants: Eli Lilly and Company, Indianapolis, IN (US); Merck Sharp & Dohme Corporation, Rahway, NJ (US)

(72) Inventors: Richard Paul BECKMANN, Avon, IN (US); Colleen Michelle MOCKBEE, McCordsville, IN (US); David Arlen SCHAER, Mamaroneck, NY (US)

(73) Assignee: Eli Lilly and Company, Indianapolis, IN (US)

(21) Appl. No.: 16/301,835

(22) PCT Filed: May 19, 2017
(86) PCT No.: PCT/US2017/033633

§ 371 Date: Nov. 15, 2018

Related U.S. Application Data
(60) Provisional application No. 62/340,197, filed on May 23, 2016.
(60) Provisional application No. 62/417,032, filed on Nov. 3, 2016.

(51) Int. Cl. A61K 38/17; A61P 35/00; A61K 31/506

(52) U.S. Cl. CPC A61K 38/1774; A61K 31/506; A61P 35/00

(57) ABSTRACT

The present invention relates to a combination of abemaciclib and pembrolizumab and methods of using the combination to treat certain disorders, such as breast cancer and non-small cell lung cancer.

The present invention relates to a combination of abemaciclib, or a pharmaceutically acceptable salt thereof, and pembrolizumab or a pembrolizumab variant and to methods of using the combination to treat certain disorders, such as non-small cell lung cancer (NSCLC) or breast cancer.

NSCLC is the most common type of lung cancer. About 85% of lung cancers are non-small cell lung cancers. Squamous cell carcinoma, adenocarcinoma, and large cell carcinoma are all subtypes of non-small cell lung cancer. The KRAS mutation is present in about 20% to 30% of patients with lung adenocarcinoma and KRAS is one of the most commonly recurrent oncogene mutations in lung cancer.

Breast cancer is the most common cancer among women worldwide. It is also the leading cause of cancer deaths among women (˜450,000/yr). Metastatic Breast Cancer (mBC) occurs when the cancer spreads beyond the breast to other parts of the body. In addition to the patients who are initially diagnosed with mBC, nearly 30% of women diagnosed with early breast cancer will eventually develop mBC. Patients diagnosed with mBC face a median survival of 2-4 years, and mBC currently remains incurable.

Abemaciclib (LY2835219), [5-(4-ethyl-piperazin-1-ylmethyl)-pyridin-2-yl]-[5-fluoro-4-(7-fluoro-3-isopropyl-2-methyl-3H-benzoimidazol-5-yl)-pyrimidin-2-yl]-amine, is a CDK inhibitor that targets the CDK4 and CDK6 cell cycle pathway, with antineoplastic activities. Abemaciclib, including salt forms, and methods of making and using this compound including for the treatment of cancer, in particular, breast cancer and lung cancer, are disclosed in WO2010/075074. Abemaciclib has also shown clinical activity in both breast cancer and NSCLC. Abemaciclib has the following structure:

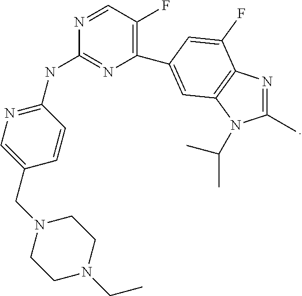 [SMILES: CCN1CCN(Cc2ccc(Nc3ncc(F)c(-c4cc(F)c5nc(C)n(C(C)C)c5c4)n3)nc2)CC1]

Pembrolizumab (Keytruda®) is a humanized IgG4 monoclonal antibody against programmed death receptor-1 (PD-1). Pembrolizumab and methods of making and using this compound are disclosed in WO2008156712. Pembrolizumab has been shown to inhibit the binding of PD-1 to PD-L1 and PD-L2, and has been tested in various clinical trials. (WO2008156712and Hamid et al., N Engl J Med (2013) 369:2). Pembrolizumab is approved by the US Food and Drug Administration (FDA) for the treatment of patients with unresectable or metastatic melanoma, patients with metastatic NSCLC whose tumors have high PD-L1 expression as determined by an FDA-approved test with no EGFR or ALK genomic tumor aberrations, and no prior systemic chemotherapy treatment, and for patients with metastatic NSCLC whose tumors express PD-L1 and who have disease progression on or after platinum-containing chemotherapy. Patients with EGFR or ALK genomic tumor aberrations should have disease progression on FDA-approved therapy for these aberrations prior to receiving pembrolizumab. Pembrolizumab is also approved for patients with recurrent or metastatic head and neck squamous cell carcinoma (HNSCC) with disease progression on or after platinum-containing chemotherapy and for adult and pediatric patients with refractory classical Hodgkin lymphoma or who have relapsed after 3 or more prior lines of therapy.

Combinations of inhibitors of CDK4 and 6 inhibitors and PD-1 inhibitors have been contemplated in the art. See, for example, WO16040880, WO16040882, and NCT02779751 and NCT02079636 (clinical trials.gov).

A cure for breast cancer and non-small cell lung cancer still remains elusive and there exists a need for more and different therapies that may prove to be effective in treating these conditions. Given the available data and the critical pathways involved in these cancers, the combination of abemaciclib and pembrolizumab may provide a new treatment option for patients.

According to an aspect of the present invention, there is presented a method of treating breast cancer, comprising administering to a patient:
(1) 200 mg of an antibody or antigen binding fragment thereof comprising three light chain complementarity determining regions (CDRs, (CDRL1, CDRL2 and CDRL3)) and three heavy chain CDRs (CDRH1, CDRH2, and CDRH3), wherein CDRL1 comprises the amino acid sequence set forth in SEQ ID NO:5, CDRL2 comprises the amino acid sequence set forth in SEQ ID NO:6, CDRL3 comprises the amino acid sequence set forth in SEQ ID NO:7, CDRH1 comprises the amino acid sequence set forth in SEQ ID NO:8, CDRH2 comprises the amino acid sequence set forth in SEQ ID NO:9, and CDRH3 comprises the amino acid sequence set forth in SEQ ID NO:10 on day 1 of a 21-day cycle, and
(2) 150 mg of abemaciclib or a pharmaceutically acceptable salt thereof twice daily on days 1-21 of the 21-day cycle or
(3) 100 mg of abemaciclib or a pharmaceutically acceptable salt thereof twice daily on days 1-21 of the 21-day cycle.

In some embodiments, the antibody or antigen binding fragment thereof comprises a light chain variable region (LCVR) amino acid sequence of SEQ ID NO: 1 and a heavy chain variable region (HCVR) amino acid sequence of SEQ ID NO: 2. In some embodiments, the antibody or antigen binding fragment thereof comprises a light chain (LC) amino acid sequence of SEQ ID NO: 3, and a heavy chain (HC) amino acid sequence of SEQ ID NO: 4. In some embodiments, the antibody is pembrolizumab. Preferably the breast cancer is Hormone Receptor postitive (HR+), human epidermal growth factor receptor 2 negative (HER2−) metastatic breast cancer. Preferably the breast cancer is Hormone Receptor positive (HR+), human epidermal growth factor receptor 2 positive (HER2+) metastatic breast cancer.

According to another aspect of the present invention, there is presented a method of treating breast cancer, comprising administering to a patient 200 mg of an antibody or antigen binding fragment thereof comprising a CDRL1 comprising the amino acid sequence set forth in SEQ ID NO:5, a CDRL2 comprising the amino acid sequence set forth in SEQ ID NO:6, a CDRL3 comprising the amino acid sequence set forth in SEQ ID

NO:7, a CDRH1 comprising the amino acid sequence set forth in SEQ ID NO:8, a CDRH2 comprising the amino acid sequence set forth in SEQ ID NO:9, and a CDRH3 comprising the amino acid sequence set forth in SEQ ID NO:10 on day 1 of a 21-day cycle and 100 mg of abemaciclib or a pharmaceutically acceptable salt thereof twice daily on days 1-21 of the 21-day cycle. In some embodiments, the antibody or antigen binding fragment thereof comprises a LCVR amino acid sequence of SEQ ID NO: 1 and a HCVR amino acid sequence of SEQ ID NO: 2. In some embodiments, the antibody or antigen binding fragment thereof comprises a light chain amino acid sequence of SEQ ID NO: 3, and a heavy chain amino acid sequence of SEQ ID NO: 4. In some embodiments, the antibody is pembrolizumab. Preferably the breast cancer is HR+, HER2− metastatic breast cancer. Preferably the breast cancer is HR+, HER2+ metastatic breast cancer.

According to another aspect of the present invention, there is presented a method of treating non-small cell lung cancer, comprising administering to a patient 200 mg of an antibody or antigen binding fragment thereof comprising a CDRL1 comprising the amino acid sequence set forth in SEQ ID NO:5, a CDRL2 comprising the amino acid sequence set forth in SEQ ID NO:6, a CDRL3 comprising the amino acid sequence set forth in SEQ ID NO:7, a CDRH1 comprising the amino acid sequence set forth in SEQ ID NO:8, a CDRH2 comprising the amino acid sequence set forth in SEQ ID NO:9, and a CDRH3 comprising the amino acid sequence set forth in SEQ ID NO:10 on day 1 of a 21-day cycle and 150 mg of abemaciclib or a pharmaceutically acceptable salt thereof twice daily on days 1-21 of the 21-day cycle. In some embodiments, the antibody or antigen binding fragment thereof comprises a LCVR amino acid sequence of SEQ ID NO: 1 and a HCVR amino acid sequence of SEQ ID NO: 2. In some embodiments, the antibody or antigen binding fragment thereof comprises a light chain amino acid sequence of SEQ ID NO: 3, and a heavy chain amino acid sequence of SEQ ID NO: 4. In some embodiments, the antibody is pembrolizumab. Preferably the non-small cell lung cancer has a (V-Ki-ras2 Kirsten rat sarcoma viral oncogene homolog) KRAS mutation and is Programmed cell death ligand 1 positive, (PD-L1+), collectively (KRAS mutant, PD-L1+). Preferably the non-small cell lung cancer is squamous.

According to another aspect of the present invention, there is presented a method of treating non-small cell lung cancer, comprising administering to a patient 200 mg of an antibody or antigen binding fragment thereof comprising a CDRL1 comprising the amino acid sequence set forth in SEQ ID NO:5, a CDRL2 comprising the amino acid sequence set forth in SEQ ID NO:6, a CDRL3 comprising the amino acid sequence set forth in SEQ ID NO:7, a CDRH1 comprising the amino acid sequence set forth in SEQ ID NO:8, a CDRH2 comprising the amino acid sequence set forth in SEQ ID NO:9, and a CDRH3 comprising the amino acid sequence set forth in SEQ ID NO:10 on day 1 of a 21-day cycle and 100 mg of abemaciclib or a pharmaceutically acceptable salt thereof twice daily on days 1-21 of the 21-day cycle. In some embodiments, the antibody or antigen binding fragment thereof comprises a LCVR amino acid sequence of SEQ ID NO: 1 and a HCVR amino acid sequence of SEQ ID NO: 2. In some embodiments, the antibody or antigen binding fragment thereof comprises a light chain amino acid sequence of SEQ ID NO: 3, and a heavy chain amino acid sequence of SEQ ID NO: 4. In some embodiements, the antibody is pembrolizumab. Preferably the non-small cell lung cancer is KRAS mutant, PD-L1+. Preferably the non-small cell lung cancer is squamous.

According to another aspect of the present invention, there is presented method of treating cancer selected from the group consisting of NSCLC or breast cancer in a patient, comprising administering to the patient an effective amount of pembrolizumab and abemaciclib, or a pharmaceutically acceptable salt thereof, wherein initial administration of the abemaciclib or the salt thereof is administered to the patient without the permbrolizumab for at least 24 hours and the abemaciclib or the salt thereof is then administered to the patient in combination with the pembrolizumab. Preferably the cancer is NSCLC. Preferably the cancer is breast cancer. Preferably the administration of the abemaciclib or the salt thereof is stopped for a period of time determined by a treating physician to alleviate side effects of the abemaciclib or the salt thereof, and/or the pembolizumab. Preferably the administration of the abemaciclib or the salt thereof is stopped after initiation of the combination with the pembrolizumab, and administration of the pembrolizumab is continued without administering the abemaciclib or the salt thereof. Preferably the abemaciclib or the salt thereof is administered orally at 150 mg approximately every 12 hours. Preferably the abemaciclib or the salt thereof is administered orally at 100 mg approximately every 12 hours.

According to another aspect of the present invention, there is presented a kit comprising an antibody or antigen binding fragment thereof comprising a CDRL1 comprising the amino acid sequence set forth in SEQ ID NO:5, a CDRL2 comprising the amino acid sequence set forth in SEQ ID NO:6, a CDRL3 comprising the amino acid sequence set forth in SEQ ID NO:7, a CDRH1 comprising the amino acid sequence set forth in SEQ ID NO:8, a CDRH2 comprising the amino acid sequence set forth in SEQ

ID NO:9, and a CDRH3 comprising the amino acid sequence set forth in SEQ ID NO:10 and abemaciclib or a pharmaceutically acceptable salt thereof, for the treatment of breast cancer. In some embodiments, the antibody or antigen binding fragment thereof comprises a LCVR amino acid sequence of SEQ ID NO: 1 and a HCVR amino acid sequence of SEQ ID NO: 2. In some embodiments, the antibody or antigen binding fragment thereof comprises a light chain amino acid sequence of SEQ ID NO: 3, and a heavy chain amino acid sequence of SEQ ID NO: 4. In some embodiments, the antibody is pembrolizumab. Preferably the breast cancer is HR+, HER2− metastatic breast cancer. Preferably the breast cancer is HR+, HER2+ metastatic breast cancer.

According to another aspect of the present invention, there is presented a kit comprising an antibody or antigen binding fragment thereof comprising a CDRL1 comprising the amino acid sequence set forth in SEQ ID NO:5, a CDRL2 comprising the amino acid sequence set forth in SEQ ID NO:6, a CDRL3 comprising the amino acid sequence set forth in SEQ ID NO:7, a CDRH1 comprising the amino acid sequence set forth in SEQ ID NO:8, a CDRH2 comprising the amino acid sequence set forth in SEQ

ID NO:9, and a CDRH3 comprising the amino acid sequence set forth in SEQ ID NO:10 and abemaciclib or a pharmaceutically acceptable salt thereof, for the treatment of non-small cell lung cancer. In some embodiments, the antibody or antigen binding fragment thereof comprises a LCVR amino acid sequence of SEQ ID NO: 1 and a HCVR amino acid sequence of SEQ ID NO: 2. In some embodiments, the antibody or antigen binding fragment thereof comprises a light chain amino acid sequence of SEQ ID NO: 3, and a heavy chain amino acid sequence of SEQ ID NO: 4. In some embodiments, the antibody is pembrolizumab. Preferably the non-small cell lung cancer is KRAS mutant, PD-L1+. Preferably the non-small cell lung cancer is squamous.

According to another aspect of the present invention, there is presented a kit comprising pembrolizumab, with one or more pharmaceutically acceptable carriers, diluents, or excipients, and abemaciclib, or a pharmaceutically acceptable salt thereof, with one or more pharmaceutically acceptable carriers, diluents, or excipients, for the treatment of breast cancer. Preferably abemaciclib, or the pharmaceutically acceptable salt thereof, is a tablet. Also preferably, abemaciclib, or a pharmaceutically acceptable salt thereof, is a capsule. Preferably the breast cancer is HR+, HER2− metastatic breast cancer. Preferably the breast cancer is HR+, HER2+ metastatic breast cancer.

According to another aspect of the present invention, there is presented a kit comprising pembrolizumab, with one or more pharmaceutically acceptable carriers, diluents, or excipients, and abemaciclib, or a pharmaceutically acceptable salt thereof, with one or more pharmaceutically acceptable carriers, diluents, or excipients, for the treatment of non-small cell lung cancer. Preferably abemaciclib, or the pharmaceutically acceptable salt thereof, is a tablet. Also preferably, abemaciclib, or a pharmaceutically acceptable salt thereof, is a capsule. Preferably the non-small cell lung cancer is KRAS mutant, PD-L1+. Preferably the non-small cell lung cancer is squamous.

According to another aspect of the present invention, there is presented a combination comprising 200 mg of pembrolizumab and 150 mg of abemaciclib, or a pharmaceutically acceptable salt thereof, for use in the treatment of breast cancer wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is

HR+, HER2+ metastatic breast cancer.

According to another aspect of the present invention, there is presented a combination comprising 200 mg of pembrolizumab and 100 mg of abemaciclib, or a pharmaceutically acceptable salt thereof, for use in the treatment of breast cancer wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

According to another aspect of the present invention, there is presented a combination comprising 200 mg of pembrolizumab and 150 mg of abemaciclib, or a pharmaceutically acceptable salt thereof, for use in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented a combination comprising 200 mg of pembrolizumab and 100 mg of abemaciclib, or a pharmaceutically acceptable salt thereof, for use in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented a combination comprising 200 mg of pembrolizumab and 150 mg of abemaciclib, or a pharmaceutically acceptable salt thereof, for use in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented a combination comprising 200 mg of pembrolizumab and 100 mg of abemaciclib, or a pharmaceutically acceptable salt thereof, for use in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented a combination comprising 200 mg of pembrolizumab and 150 mg of abemaciclib, or a pharmaceutically acceptable salt thereof, for use in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

According to another aspect of the present invention, there is presented a combination comprising 200 mg of pembrolizumab and 100 mg of abemaciclib, or a pharmaceutically acceptable salt thereof, for use in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

According to another aspect of the present invention, there is presented 200 mg pembrolizumab for use in combination with 150 mg abemaciclib,or a pharmaceutically acceptable salt thereof, in the treatment of breast cancer wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

According to another aspect of the present invention, there is presented 200 mg pembrolizumab for use in combination with 100 mg abemaciclib,or a pharmaceutically acceptable salt thereof, in the treatment of breast cancer wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

According to another aspect of the present invention, there is presented 200 mg pembrolizumab for use in combination with 150 mg abemaciclib,or a pharmaceutically acceptable salt thereof, in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented 200 mg pembrolizumab for use in combination with 100 mg abemaciclib,or a pharmaceutically acceptable salt thereof, in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented 200 mg pembrolizumab for use in combination with 150 mg abemaciclib,or a pharmaceutically acceptable salt thereof, in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented 200 mg pembrolizumab for use in combination with 100 mg abemaciclib,or a pharmaceutically acceptable salt thereof, in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented 200 mg pembrolizumab for use in combination with 150 mg abemaciclib,or a pharmaceutically acceptable salt thereof, in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

According to another aspect of the present invention, there is presented 200 mg pembrolizumab for use in combination with 100 mg abemaciclib,or a pharmaceutically acceptable salt thereof, in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

According to another aspect of the present invention, there is presented 150 mg abemaciclib, or a pharmaceutically acceptable salt thereof, for use combination with 200 mg pembrolizumab in the treatment of breast cancer wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

According to another aspect of the present invention, there is presented 100 mg abemaciclib, or a pharmaceutically acceptable salt thereof, for use combination with 200 mg pembrolizumab in the treatment of breast cancer wherein the pembrolizumab is administered on day 1 of a 21-day cycle and the abemaciclib or the salt thereof is administered twice daily on days 1-21 of the 21-day cycle. Preferably the breast cancer is

HR+, HER2− metastatic breast cancer. Preferably the breast cancer is HR+, HER2+ metastatic breast cancer.

According to another aspect of the present invention, there is presented 150 mg abemaciclib, or a pharmaceutically acceptable salt thereof, for use combination with 200 mg pembrolizumab in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented 100 mg abemaciclib, or a pharmaceutically acceptable salt thereof, for use combination with 200 mg pembrolizumab in the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

According to another aspect of the present invention, there is presented 150 mg abemaciclib, or a pharmaceutically acceptable salt thereof, for use combination with 200 mg pembrolizumab in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented 100 mg abemaciclib, or a pharmaceutically acceptable salt thereof, for use combination with 200 mg pembrolizumab in the treatment of non-small cell lung cancer wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

According to another aspect of the present invention, there is presented 150 mg abemaciclib, or a pharmaceutically acceptable salt thereof, for use combination with 200 mg pembrolizumab in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

According to another aspect of the present invention, there is presented 100 mg abemaciclib, or a pharmaceutically acceptable salt thereof, for use combination with 200 mg pembrolizumab in the treatment of non-small cell lung cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

The present invention also provides for use of 200 mg pembrolizumab in the manufacture of a medicament for the treatment of breast cancer wherein the medicament is to be administered in combination with 150 mg abemaciclib, or a pharmaceutically acceptable salt thereof wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

The present invention also provides for use of 200 mg pembrolizumab in the manufacture of a medicament for the treatment of breast cancer wherein the medicament is to be administered in combination with 100 mg abemaciclib, or a pharmaceutically acceptable salt thereof wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

The present invention also provides for use of 200 mg pembrolizumab in the manufacture of a medicament for the treatment of breast cancer wherein the medicament is to be administered in combination with 150 mg abemaciclib, or a pharmaceutically acceptable salt thereof wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

The present invention also provides for use of 200 mg pembrolizumab in the manufacture of a medicament for the treatment of breast cancer wherein the medicament is to be administered in combination with 100 mg abemaciclib, or a pharmaceutically acceptable salt thereof wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

The present invention also provides for use of 200 mg pembrolizumab in the manufacture of a medicament for the treatment of non-small cell lung cancer wherein the medicament is to be administered in combination with 150 mg abemaciclib, or a pharmaceutically acceptable salt thereof wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

The present invention also provides for use of 200 mg pembrolizumab in the manufacture of a medicament for the treatment of non-small cell lung cancer wherein the medicament is to be administered in combination with 100 mg abemaciclib, or a pharmaceutically acceptable salt thereof wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

The present invention also provides for use of 200 mg pembrolizumab in the manufacture of a medicament for the treatment of non-small cell lung cancer wherein the medicament is to be administered in combination with 150 mg abemaciclib, or a pharmaceutically acceptable salt thereof wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

The present invention also provides for use of 200 mg pembrolizumab in the manufacture of a medicament for the treatment of non-small cell lung cancer wherein the medicament is to be administered in combination with 100 mg abemaciclib, or a pharmaceutically acceptable salt thereof wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

The present invention also provides for use of 150 mg abemaciclib or a pharmaceutically acceptable salt thereof in the manufacture of a medicament for the treatment of breast cancer wherein the medicament is to be administered in combination with 200 mg pembrolizumab wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

The present invention also provides for use of 100 mg abemaciclib or a pharmaceutically acceptable salt thereof in the manufacture of a medicament for the treatment of breast cancer wherein the medicament is to be administered in combination with 200 mg pembrolizumab wherein the pembrolizumab is administered on day 1 of a 21-day cycle and the abemaciclib or the salt thereof is administered twice daily on days 1-21 of the 21-day cycle. Preferably the breast cancer is HR+, HER2− metastatic breast cancer. Preferably the breast cancer is HR+, HER2+ metastatic breast cancer.

The present invention also provides for use of 150 mg abemaciclib or a pharmaceutically acceptable salt thereof in the manufacture of a medicament for the treatment of breast cancer wherein the medicament is to be administered in combination with 200 mg pembrolizumab wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2− metastatic breast cancer. Preferably the breast cancer is HR+, HER2+ metastatic breast cancer.

The present invention also provides for use of 100 mg abemaciclib or a pharmaceutically acceptable salt thereof in the manufacture of a medicament for the treatment of breast cancer wherein initial administration of the abemaciclib or the salt thereof is administered to the patient without the permbrolizumab for at least 24 hours and the abemaciclib or the salt thereof is then administered to the patient in combination with the pembrolizumab. Preferably the breast cancer is HR+, HER2−metastatic breast cancer. Preferably the breast cancer is HR+, HER2+ metastatic breast cancer.

The present invention also provides for use of 150 mg abemaciclib or a pharmaceutically acceptable salt thereof in the manufacture of a medicament for the treatment of non-small cell lung cancer wherein the medicament is to be administered in combination with 200 mg pembrolizumab wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

The present invention also provides for use of 100 mg abemaciclib or a pharmaceutically acceptable salt thereof in the manufacture of a medicament for the treatment of non-small cell lung cancer wherein the medicament is to be administered in combination with 200 mg pembrolizumab wherein the pembrolizumab is administered on day 1 of a 21-day cycle and the abemaciclib or the salt thereof is administered twice daily on days 1-21 of the 21-day cycle. Preferably the non-small cell lung cancer is KRAS mutant, PD-L1+. Preferably the non-small cell lung cancer is squamous.

The present invention also provides for use of 150 mg abemaciclib or a pharmaceutically acceptable salt thereof in the manufacture of a medicament for the treatment of non-small cell lung cancer wherein the medicament is to be administered in combination with 200 mg pembrolizumab wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

The present invention also provides for use of 100 mg abemaciclib or a pharmaceutically acceptable salt thereof in the manufacture of a medicament for the treatment of non-small cell lung cancer wherein the medicament is to be administered in combination with 200 mg pembrolizumab wherein initial administration of the abemaciclib or the salt thereof is administered to the patient without the permbrolizumab for at least 24 hours and the abemaciclib or the salt thereof is then administered to the patient in combination with the pembrolizumab. Preferably the non-small cell lung cancer is KRAS mutant, PD-L1+. Preferably the non-small cell lung cancer is squamous.

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes amino acid sequences of CDRs and framework regions (FRs).

As used herein, the term “heavy chain variable region” “HCVR” refers to a portion of a heavy chain of an antibody molecule that includes amino acid sequences of CDRs and FRs.

As used herein, “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv and multispecific antibodies formed from antibody fragments.

As used herein, the term “kit” refers to a package comprising at least two separate containers, wherein a first container contains abemaciclib, or a pharmaceutically acceptable salt thereof, and a second container contains pembrolizumab. A “kit” may also include instructions to administer all or a portion of the contents of these first and second containers to a cancer patient, preferably a non-small cell lung cancer patient or a breast cancer patient.

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the term, “partial response” refers to a decrease in the size of a tumor, or in the extent of cancer in the body, in response to treatment.

As used herein, the term “patient” refers to a mammal, preferably a human.

As used herein, the terms “cancer” refers to or describe the physiological condition in patients that is typically characterized by unregulated cell proliferation. Included in this definition are benign and malignant cancers.

The route of administration may be varied in any way, limited by the physical properties of the drugs and the convenience of the patient and the caregiver. Preferably, abemaciclib, or a pharmaceutically acceptable salt thereof, is formulated orally in a capsule or tablet and pembrolizumab is formulated for parenteral administration, such as intravenous or subcutaneous administration.

As used herein, the phrase “in combination with” refers to the administration of abemaciclib, or a pharmaceutically acceptable salt thereof, and pembrolizumab or a pembrolizumab variant.

### EXAMPLE 1

### Concurrent Dosing of Abemaciclib with a PD-L1 Checkpoint Immunotherapy Results in Improved Anti-Tumor Efficacy Compared to PD-L1 Monotherapy

CT26 is also a well-known model for immunotherapy. Studies have shown it to be sensitive to inhibitors of a co-inhibitory immune checkpoint including, CTLA-4, PD-1, PD-L1 blockade. Established sensitivity of CT26, and well known range of therapeutic efficacy of immunotherapeutic intervention make it an appropriate model for screening the activity of checkpoint immunotherapies (Grosso and Jure-Kunkel, Cancer Immunity Vol. 13, p. 5, January 2013) (Duraiswamy et al, Cancer Research, Jun. 15, 2013 73; 3591).

CT26 is a murine model, and accordingly, antibodies used in the CT26 model must bind the mouse target protein. To model co-inhibitory immune checkpoint therapy, in particular blockade of PD-L1/PD-1 mediated inhibition, anti-murine PD-L1 clone, 178G7 (Known herein in the examples as: anti-PD-L1 antibody or anti-PD-L1 therapy or anti-PD-L1), is produced. The HC of 178G7 is SEQ ID NO: 11, and the LC of 178G7 is SEQ ID NO: 12. 178G7 blocks PD-1 binding to PD-L1. In particular, 178G7 is used as a a surrogate agents that prevent PD-1 mediated inhibition by PD-L1. as both 178G7 and pembrolizumab block the interaction of of PD-1 and PD-L1. 178G7 competes with previously identified surrogate antibody 10F.9G2 known to block PD-L1/PD-1 interaction and is a known surrogate for anti-human antibodies in the clinic (Eppihimer et al. Microcirculation 2002:9(2):133).

Abemaciclib is combined concurrently from the initiation of therapy with the anti-PD-L1 checkpoint immunotherapy to see if it can improve abemaciclib monotherapy. This would mimic a potential clinical setting where an anti-PD-L1 antibody therapy would be used to improve the response to abemaciclib. To determine the ability of an anti-PD-L1 checkpoint immunotherapy to enhance the efficacy of abemaciclib, 50 mg/kg of abemaciclib is tested in combination with an anti-PD-L1 antibody on the growth of CT26 syngeneic tumors, CT26 tumor cells are cultured for implantation in medium (RPMI1640; 10% fetal bovine serum; 1 mM sodium pyruvate; 2 mM L-glutamine). Cells are passaged twice a week before harvesting and then Balb/C mice are implanted in the rear flank with 1×10⁶ CT26 cells in phosphate buffered saline. Tumors are allowed to grow for 6 days to between 50-100 mm³ in size before treatment. At 6 days following implantation (study day 6) groups of 5-10 animals total are treated with either vehicle (1% hydroxyethyl cellulose (HEC) in 25 mM phosphate buffer, pH 2) 50 mg/kg abemaciclib for 21 days in monotherapy, anti-PD-L1 antibody therapy alone (178G7 flat dosed 500 μg/dose 1 time per week, for 3 doses sufficient to saturate PD-L1 for 3 weeks), or a concurrent combination of 50 mg/kg abemaciclib for 21 days combined with 3 weekly doses of the anti-PD-L1 antibody therapy. If tumor burden becomes greater than 2500 mm³ animals are sacrificed due to progressive disease, or at lower volume if the size of the tumor suggests that they will surpass 2500 mm³ before the next scheduled measurement in accordance with animal use policies.

### Tumor Growth Analysis

Relative changes in tumor volume compared to control are analyzed at day 24, at the point where the most animals remained in all groups for proper statistical comparison. Relative changes in tumor volume (% ΔT/C) are calculated using the formula % ΔT/C=100×ΔT/ΔC, whereby T=mean tumor volume of the compound treated group, ΔT=mean tumor volume of the compound treated group minus the mean tumor volume on the baseline day, C=mean tumor volume of the control (vehicle) group, and ΔC=mean tumor volume of the control group minus the mean tumor volume on the baseline day. The baseline day is the nearest day just prior to the initiation of treatment.

As shown in Table 1, treatment with abemaciclib caused a significant tumor growth inhibition in monotherapy compared to control (%T/C=22.3%). Anti-PD-L1 antibody monotherapy treatment also resulted in a growth delay compared to control, which trended toward significance (%T/C=56.1). Concurrent combination of abemaciclib with the anti-PD-L1 antibody caused a significant growth delay compared to control (%T/C=25.4%); however this was not significantly different from abemaciclib monotherapy.

In the above example, concurrent dosing from the initation of therapy of both abemaciclib and the anti-PD-L1 results in improved effacacy compared to PD-L1 monotherapy, but there was no apparent difference in the anti-tumor response compared to abemaciclib alone. This data demonstrates that abemaciclib can be added to PD-L1/PD-1 blockade therapy without reducing the efficacy of PD-L1/PD-1 blockade, and supports that cell cycle inhibitors can be combined with immunotherapy checkpoint blockade.

### Example 2

A Phase 1 study is being conducted to evaluate the safety and efficacy of abemaciclib in combination with pembrolizumab in participants with advanced NSCLC or HR+, HER2− breast cancer. Abemaciclib is given orally every 12 hours on days 1 to 21 of each 21 day cycle at a dose of 150 mg in combination with pembrolizumab given intravenously on day 1 of each 21 day cycle at a dose of 200 mg.

At an unofficial preliminary evaluation, thirteen of the breast cancer patients and eighteen of the NSCLC patients were far along enough to be assessed. Of the assessed breast cancer patients, two of the patients had confirmed partial responses, four of the patients had stable disease, two of the patients had progressive disease, one of the patients was removed for an adverse event, and four of the patients were not reported. Of the assessed NSCLC patients, eight of the patients had stable disease, two of the patients had progressive disease, and eight of the patients were deemed nonevaluable.

SEQUENCE LISTING

SEQ ID NO: 1 (LCVR of pembrolizumab)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK
SEQ ID NO: 2 (HCVR of pembrolizumab)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS
SEQ ID NO: 3 (LC of pembrolizumab)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 4 (HC of pembrolizumab)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 5 (CDRL1 of pembrolizumab)
RASKGVSTSGYSYLH
SEQ ID NO: 6 (CDRL2 of pembrolizumab)
LASYLES
SEQ ID NO: 7 (CDRL3 of pembrolizumab)
QHSRDLPLT
SEQ ID NO: 8 (CDRH1 of pembrolizumab)
NYYMY
SEQ ID NO: 9 (CDRH2 of pembrolizumab)
GINPSNGGTNFNEKFK
SEQ ID NO: 10 (CDRH3 of pembrolizumab)
RDYRFDMGFDY
SEQ ID NO: 11 (HC of 178G7)
QVQLQQSGADLAKPGSSVKISCKASGYNFNSYYINWIKQTTGQGLEYIGY
INTVSGTTKYSEKFKGKATLTVDKSSSTAFMQLSSLTPDDSAVYYCARGT
IVLDDYWGQGVKVTVSSAETTAPSVYPLAPGTALKSNSMVTLGCLVKGYF
PEPVTVTWNSGALSSGVHTFPAVLQSGLYTLTSSVTVPSSTWPSQTVTCN
VAHPASSTKVDKKIVPRNCGGDCKPCICTGSEVSSVFIFPSKPKDVLTIT
LTPKVTCVVVDISQDDPEVHFSWFVDDVEVHTAQTRPPEEQFNSTFRSVS
ELPILHQDWLNGRTFRCKVTSAAFPSPIEKTISKPEGRTQVPHVYTMSPT
KEEMTQNEVSITCMVKGFYPPDIYVEWQMNGQPQENYKNTPPTMDTDGSY
FLYSKLNVKKEKWQQGNTFTCSVLHEGLHNHHTEKSLSHSPGK
SEQ ID NO: 12 (LC of 178G7)
DIVMTQTPSSQAVSAGEKVTMSCKSSQSLLYNEKKKNYLAWYQQKPGQSP
KLLIYWASTRESGVPDRFLGSGSGTDFTLTINSVQAEDLAVYYCQQSYDF
PRTFGGGTKLELKRDAAPTVSIFPPSTEQLATGGASVVCLMNNFYPRDIS
VKWKIDGTERRDGVLDSVTDQDSKDSTYSMSSTLSLTKADYESHNLYTCE
VVHKTSSSPVVKSFNRNEC

We claim:

1. A method of treating breast cancer, comprising administering to a patient 200 mg of an anti-PD-1 antibody or antigen binding fragment thereof comprising three light chain CDRs (CDRL1, CDRL2 and CDRL3) and three heavy chain CDRs (CDRH1, CDRH2, and CDRH3), wherein CDRL1 comprises the amino acid sequence set forth in SEQ ID NO:5, CDRL2 comprises the amino acid sequence set forth in SEQ ID NO:6, CDRL3 comprises the amino acid sequence set forth in SEQ ID NO:7, CDRH1 comprises the amino acid sequence set forth in SEQ ID NO:8, CDRH2 comprises the amino acid sequence set forth in SEQ ID NO:9, and CDRH3 comprises the amino acid sequence set forth in SEQ ID NO:10 on day 1 of a 21-day cycle and 150 mg of abemaciclib or a pharmaceutically acceptable salt thereof twice daily on days 1-21 of the 21-day cycle.

2. A method of treating breast cancer, comprising administering to a patient 200 mg of an anti-PD-1 antibody or antigen binding fragment thereof comprising three light chain CDRs (CDRL1, CDRL2 and CDRL3) and three heavy chain CDRs (CDRH1, CDRH2, and CDRH3), wherein CDRL1 comprises the amino acid sequence set forth in SEQ ID NO:5, CDRL2 comprises the amino acid sequence set forth in SEQ ID NO:6 , CDRL3 comprises the amino acid sequence set forth in SEQ ID NO:7, CDRH1 comprises the amino acid sequence set forth in SEQ ID NO:8, CDRH2 comprises the amino acid sequence set forth in SEQ ID NO:9, and CDRH3 comprises the amino acid sequence set forth in SEQ ID NO:10 in combination with 150 mg of abemaciclib or a pharmaceutically acceptable salt thereof wherein initial administration of the abemaciclib or the salt thereof is administered to the patient without the antibody or the antigen binding fragment thereof for at least 24 hours and the abemaciclib or the salt thereof is then administered to the patient in combination with the antibody or the antigen binding fragment thereof.

3. The method according to claim 1, wherein the antibody or antigen binding fragment thereof comprises a light chain variable region (LCVR) amino acid sequence of SEQ ID NO:1 and a heavy chain variable region (HCVR) amino acid sequence of SEQ ID NO: 2.

4. (canceled)

5. (canceled)

6. (canceled)

7. The method according to claim 1 wherein the breast cancer is HR+, HER2− metastatic breast cancer.

8. (canceled)

9. A method of treating non-small cell lung cancer, comprising administering to a patient 200 mg of an anti-PD-1 antibody or antigen binding fragment thereof comprising three light chain CDRs (CDRL1, CDRL2 and CDRL3) and three heavy chain CDRs (CDRH1, CDRH2, and CDRH3), wherein CDRL1 comprises the amino acid sequence set forth in SEQ ID NO:5, CDRL2 comprises the amino acid sequence set forth in SEQ ID NO:6, CDRL3 comprises the amino acid sequence set forth in SEQ ID NO:7, CDRH1 comprises the amino acid sequence set forth in SEQ ID NO:8, CDRH2 comprises the amino acid sequence set forth in SEQ ID NO:9, and CDRH3 comprises the amino acid sequence set forth in SEQ ID NO:10 on day 1 of a 21-day cycle and 150 mg of abemaciclib or a pharmaceutically acceptable salt thereof twice daily on days 1-21 of the 21-day cycle.

10. A method of treating non-small cell lung cancer, comprising administering to a patient 200 mg of an anti-PD-1 antibody or antigen binding fragment thereof comprising three light chain CDRs (CDRL1, CDRL2 and CDRL3) and three heavy chain CDRs (CDRH1, CDRH2, and CDRH3), wherein CDRL1 comprises the amino acid sequence set forth in SEQ ID NO:5, CDRL2 comprises the amino acid sequence set forth in SEQ ID NO:6, CDRL3 comprises the amino acid sequence set forth in SEQ ID NO:7, CDRH1 comprises the amino acid sequence set forth in SEQ ID NO:8, CDRH2 comprises the amino acid sequence set forth in SEQ ID NO:9, and CDRH3 comprises the amino acid sequence set forth in SEQ ID NO:10 in combination with 150 mg of abemaciclib or a pharmaceutically acceptable salt thereof wherein initial administration of the abemaciclib or the salt thereof is administered to the patient without the antibody or the antigen binding fragment thereof for at least 24 hours and the abemaciclib or the salt thereof is then administered to the patient in combination with the antibody or the antigen binding fragment thereof

11. The method according to claim 9, wherein the antibody or antigen binding fragment thereof comprises a light chain variable region (LCVR) amino acid sequence of SEQ ID NO:1 and a heavy chain variable region (HCVR) amino acid sequence of SEQ ID NO: 2.

12. (canceled)

13. (canceled)

14. (canceled)

15. The method according to claim 9 wherein the non-small cell lung cancer is KRAS mutant, PD-L1+.

16. The method according to claim 9 wherein the non-small cell lung cancer is squamous.

17. The method according to claim 1 wherein the antibody comprises a light chain amino acid sequence of SEQ ID NO: 3, and a heavy chain amino acid sequence of SEQ ID NO: 4.

18. The method according to claim 1 where the antibody is pembrolizumab.

18-60. (canceled)

61. The method according to 2, wherein the antibody or antigen binding fragment thereof comprises a light chain variable region (LCVR) amino acid sequence of SEQ ID NO:1 and a heavy chain variable region (HCVR) amino acid sequence of SEQ ID NO: 2.

62. The method according to claim 2 wherein the breast cancer is HR+, HER2− metastatic breast cancer.

63. The method according to claim 3 wherein the breast cancer is HR+, HER2− metastatic breast cancer.

64. The method according to claim 61 wherein the breast cancer is HR+, HER2− metastatic breast cancer.

65. The method according to claim 10, wherein the antibody or antigen binding fragment thereof comprises a light chain variable region (LCVR) amino acid sequence of SEQ ID NO:1 and a heavy chain variable region (HCVR) amino acid sequence of SEQ ID NO: 2.

66. The method according to claim 10 wherein the non-small cell lung cancer is KRAS mutant, PD-L1+.

67. The method according to claim 11 wherein the non-small cell lung cancer is KRAS mutant, PD-L1+.

68. The method according to claim 65 wherein the non-small cell lung cancer is KRAS mutant, PD-L1+.

69. The method according to claim 10 wherein the non-small cell lung cancer is squamous.

70. The method according to claim 11 wherein the non-small cell lung cancer is squamous.

71. The method according to claim 65 wherein the non-small cell lung cancer is squamous.

72. The method according to claim 2 wherein the antibody comprises a light chain amino acid sequence of SEQ ID NO: 3, and a heavy chain amino acid sequence of SEQ ID NO: 4.

73. The method according to claim 2 where the antibody is pembrolizumab.

74. The method according to claim 9 wherein the antibody comprises a light chain amino acid sequence of SEQ ID NO: 3, and a heavy chain amino acid sequence of SEQ ID NO: 4.

75. The method according to claim 9 where the antibody is pembrolizumab.

76. The method according to claim 10 wherein the antibody comprises a light chain amino acid sequence of SEQ ID NO: 3, and a heavy chain amino acid sequence of SEQ ID NO: 4.

77. The method according to claim 10 where the antibody is pembrolizumab.


SEQUENCE LISTING (12 sequences)

SEQ ID NO: 1 - protein, 111 aa, Artificial Sequence [sequence omitted]

SEQ ID NO: 2 - protein, 120 aa, Artificial Sequence [sequence omitted]

SEQ ID NO: 3 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 15 aa, Artificial Sequence
  Synthetic Construct
RASKGVSTSGYSYLH

SEQ ID NO: 6 - protein, 7 aa, Artificial Sequence
  Synthetic Construct
LASYLES

SEQ ID NO: 7 - protein, 9 aa, Artificial Sequence
  Synthetic Construct
QHSRDLPLT

SEQ ID NO: 8 - protein, 5 aa, Artificial Sequence
  Synthetic Construct
NYYMY

SEQ ID NO: 9 - protein, 16 aa, Artificial Sequence
  Synthetic Construct
GINPSNGGTNFNEKFK

SEQ ID NO: 10 - protein, 11 aa, Artificial Sequence
  Synthetic Construct
RDYRFDMGFDY

SEQ ID NO: 11 - protein, 443 aa, Artificial Sequence
  Synthetic Construct
QVQLQQSGADLAKPGSSVKISCKASGYNFNSYYINWIKQTTGQGLEYIGYINTVSGTTKYSEKFKGKATLTVDKSSSTAFMQLSSLTPDDSAVYYCARGTIVLDDYWGQGVKVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPSKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 12 - protein, 220 aa, Artificial Sequence
  Synthetic Construct
DIVMTQTPSSQAVSAGEKVTMSCKSSQSLLYNEKKKNYLAWYQQKPGQSPKLLIYWASTRESGVPDRFLGSGSGTDFTLTINSVQAEDLAVYYCQQSYDFPRTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC